(19) United States
(12) Patent Application Publication
(10) Pub. No.: US 2022/0025426 A1
(43) Pub. Date: Jan. 27, 2022

(54) METHOD OF IDENTIFYING PROPERTIES OF MICROORGANISMS USING ODEP FORCE

(71) Applicants: Jang-Jih Lu, Taipei City (TW); Min-Hsien Wu, Kaohsiung City (TW); Hsin-Yao Wang, Chiayi City (TW)

(72) Inventors: Jang-Jih Lu, Taipei City (TW); Min-Hsien Wu, Kaohsiung City (TW); Hsin-Yao Wang, Chiayi City (TW)

(73) Assignee: Chang Gung University, Taoyuan City (TW)

(21) Appl. No.: 17/168,328

(22) Filed: Feb. 5, 2021

(30) Foreign Application Priority Data
Jul. 24, 2020 (TW) 109125047

(51) Int. Cl. C12Q 1/04

(52) U.S. Cl. CPC C12Q 1/04

(57) ABSTRACT

A method of identifying properties of microorganisms using ODEP force includes obtaining a sample solution of microorganisms including microorganisms having predetermined properties; pre-processing the sample solution of microorganisms to obtain a sample solution of microorganisms to be tested having predetermined electrical properties; placing the sample solution of microorganisms to be tested on a channel of a chip member; activating a light image module having combinations of different strengths of ODEP force to analyze the sample solution of microorganisms to be tested on the channel in which microorganisms in the sample solution of microorganisms to be tested are configured to generate a predetermined distribution and a predetermined presentation due to the predetermined electrical properties; and identifying properties of the microorganisms based on the predetermined distribution and the predetermined presentation.

### BACKGROUND OF THE INVENTION

### 1. Field of the Invention

The invention relates to a method of identifying properties of microorganisms and more particularly to a method of identifying properties of microorganisms using different strengths of optically-induced dielectrophoresis (ODEP) force.

### 2. Description of Related Art

Conventionally, clinical tests of microorganisms are widely used by physicians in administering medicine and by nurses in taking care of infected patients. Optimum therapy and medicine can be provided based on different properties of microorganisms. Thus, illness can be precisely and effectively cured. Conventional properties include drug resistance, toxicity, species and sub-species.

However, the conventional clinical tests of microorganisms cannot quickly identify the above properties of microorganisms. While nucleic acid testing can precisely identify properties of microorganisms, it is disadvantageous due to high cost, longer testing time, and imprecise clinical information. Taking drug resistance as an example, antibiotics susceptibility test (AST) plays a big role in healing infected patients and the use of antibiotics. The conventional art utilizes paper discs, micro-dilution, or e-tests for confirmation. Further, the AST relies on culturalization which is a time consuming process. Furthermore, the longer time the AST takes the less precise the clinical therapy will be.

Thus, the need for improvement still exists.

### SUMMARY OF THE INVENTION

It is therefore one object of the invention to provide a method of identifying properties of microorganisms using ODEP force, comprising the steps of obtaining a sample solution of microorganisms including a plurality of microorganisms having predetermined properties; pre-processing the sample solution of microorganisms to obtain a sample solution of microorganisms to be tested having predetermined electrical properties; placing the sample solution of microorganisms to be tested on a channel of a chip member; activating a light image module having combinations of different strengths of ODEP force to analyze the sample solution of microorganisms to be tested on the channel wherein the microorganisms in the sample solution of microorganisms to be tested are configured to generate a predetermined distribution and a predetermined presentation due to the predetermined electrical properties; and identifying properties of the microorganisms based on the predetermined distribution and the predetermined presentation.

The invention has the following advantages and benefits: Easy operation. Agility. Higher sensitivity, resolution and precision. It does not rely on the culturalization of microorganisms so that information of highly precise properties of microorganisms can be provided within several hours. This is a great improvement in comparison with the conventional method of identifying properties of microorganisms taking several days.

The above and other objects, features and advantages of the invention will become apparent from the following detailed description taken with the accompanying drawings.

### BRIEF DESCRIPTION OF THE DRAWINGS

FIG. 1 is a flow chart of a method of identifying properties of microorganisms using ODEP force according to the invention;

FIG. 2 is an exploded view of a chip member according to the invention;

FIG. 3 schematically depicts the chip member and a projection device according to the invention in operation;

FIGS. 4A to 4H schematically depict the sample solution of microorganisms to be tested how to flow into the chip member and the light image module how to act on both the sample solution of microorganisms to be tested and the chip member respectively; and

FIG. 5 is a table showing a result of analyzing distribution and presentation of the sample solution of microorganisms to be tested which has been acted by the light image module of FIGS. 4A to 4H.

### DETAILED DESCRIPTION OF THE INVENTION

In the invention, ODEP takes photoconductive material as a substrate (i.e., chip member). A virtual electrode is formed by controlling light images formed by projecting light from a light source under the chip member. Thus, an uneven electric field of a specific range is induced to polarize an object above the chip member and move same (i.e., dielectrophoresis (DEP)). A particle in the uneven electric field can be polarized to induce an electric dipole moment. An ODEP force is generated when extent of the polarization of the particle is different from that of surrounding solution.

The invention utilizes the method of adjusting light images to generate ODEP forces having different strengths such that specific microorganisms having properties shows different distributions and presentations due to specific electrical properties. As a result, it is possible of identifying properties of microorganisms using the different distributions and presentations.

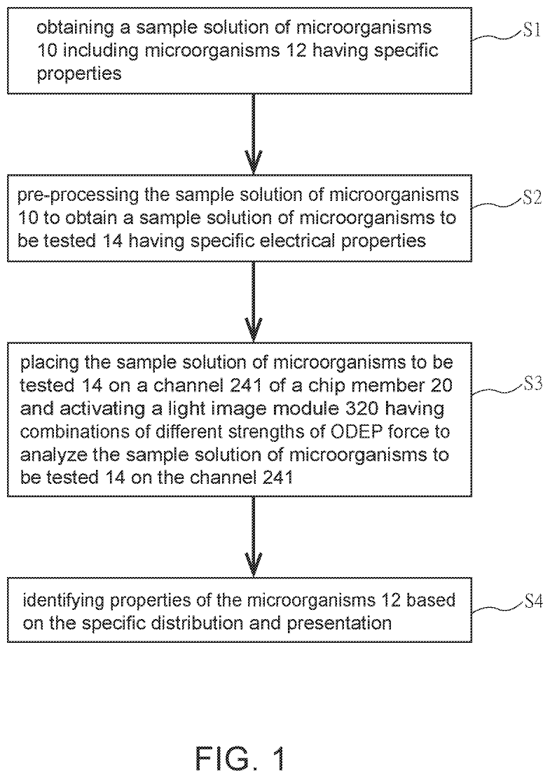

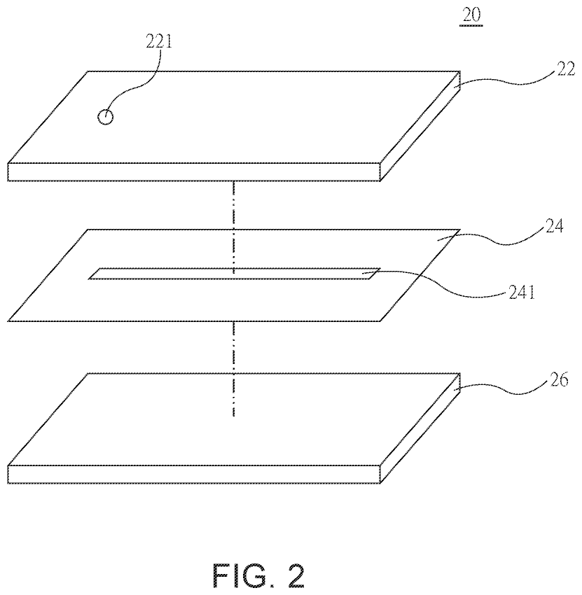

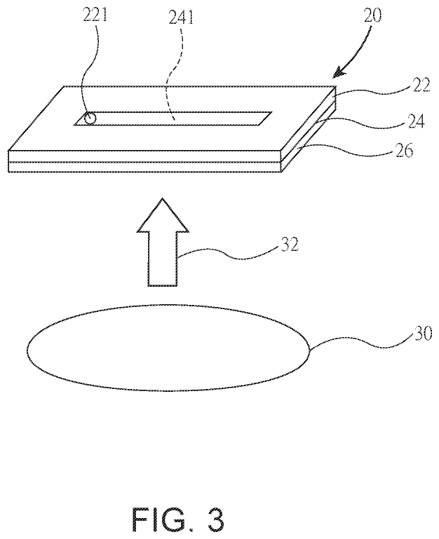

Referring to FIG. 1, a flow chart of a method of identifying properties of microorganisms using ODEP force in accordance with the invention is illustrated. The method is discussed in conjunction with FIGS. 2 and 3. The method comprises the steps of (S1) obtaining a sample solution of microorganisms 10 including a plurality of microorganisms 12 having specific properties in which the specific properties include species or sub-species of the microorganisms 12, drug resistance, toxicity, and metabolic properties; and the sample solution of microorganisms 10 is obtained by culturing blood, urine, saliva, sweat, feces, pleural fluid, ascites, or cerebrospinal fluid; (S2) pre-processing the sample solution of microorganisms 10 to obtain a sample solution of microorganisms to be tested 14 having specific electrical properties in which the pre-processing step involves culturing microorganisms, contact force, radiation, light wave, acoustic wave, shock wave, heating, freezing, electrical wave, magnetic wave, medicine or any combination thereof; (S3) placing the sample solution of microorganisms to be tested 14 on a channel 241 of a chip member 20 and activating a light image module 320 having combinations of different strengths of ODEP force to analyze the sample solution of microorganisms to be tested 14 on the channel 241 in which the light image module 320 includes a plurality of light images, and operations of the light image module 320 are achieved by adjusting a moving speed of the light images, a rotational speed of the light images, a spectral flux of the light images, and a shape of the light image, applying a frequency of alternating current (AC) to the chip member 20, applying a voltage of AC to the chip member 20, or any combination thereof; and the microorganisms 12 in the sample solution of microorganisms to be tested 14 may generate a specific distribution and presentation due to specific electrical properties; and (S4) identifying properties of the microorganisms 12 based on the specific distribution and presentation in which the microorganisms 12 are bacteria, molds, rickettsia or viruses.

Referring to FIG. 2, the chip member 20 includes, from top to bottom, a cover 22, a channel layer 24, and a photoconductive layer 26. The channel layer 24 includes the channel 241. The cover 22 includes a sample inlet 221 aligned with one end of the channel 241. The cover 22 is formed of indium-tin glass substrate. The channel layer 24 is formed of bio-compatible membrane. The photoconductive layer 26 is formed of photoconductive material.

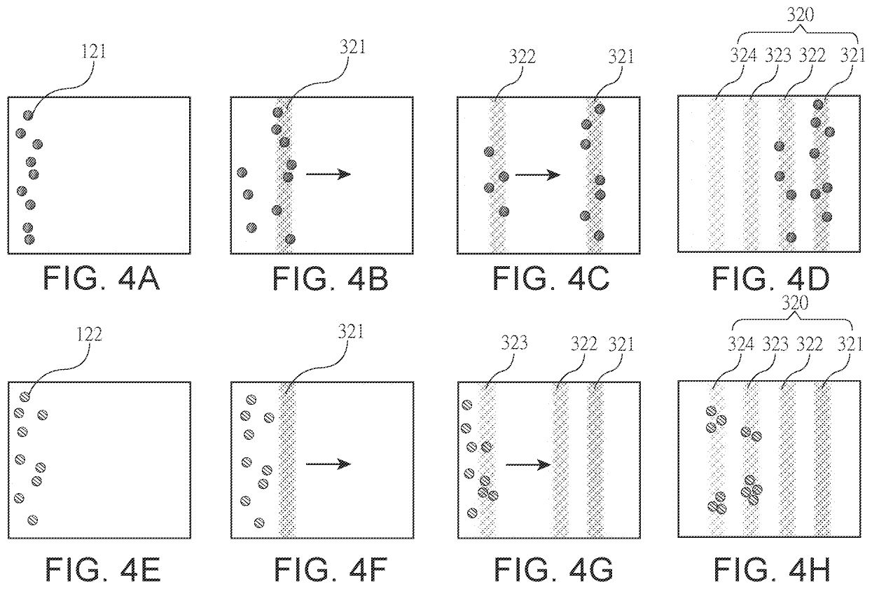

Referring to FIGS. 3 and 4 in conjunction with step S3 of FIG. 1, the chip member 20 includes the cover 22, the channel layer 24, and the photoconductive layer 26 (see FIG. 3). Further, the sample solution of microorganisms to be tested 14 is flowed through the sample inlet 221. A projection device 30 is provided under the photoconductive layer 26.

The projection device 30 may project light 32 to the photoconductive layer 26 of the chip member 20 to form light images 321, 322, 323 and 324 as shown in FIG. 4. Next, the sample solution of microorganisms to be tested 14 on the channel 241 is analyzed by adjusting a moving speed of the light images 321, 322, 323 and 324, a rotational speed of the light images 321, 322, 323 and 324, a spectral flux of the light images 321, 322, 323 and 324, and a shape of the light image 321, 322, 323 and 324, applying a frequency of AC to the chip member 20, applying a voltage of AC to the chip member 20, or any combination thereof.

As shown in FIGS. 4A to 4H specifically, it schematically depicts the sample solution of microorganisms to be tested 14 flowing into the chip member 20 and the light image module 320 acting on both the chip member 20 and the sample solution of microorganisms to be tested 14. Taking drug resistance as an example, after the pre-processed sample solution of microorganisms to be tested 14 has flowed into the chip member 20, distribution of drug-resistant microorganisms 121 is shown in FIG. 4A and distribution of drug-consumable microorganisms 122 is shown in FIG. 4E respectively.

As drug-resistant microorganisms 121 moving from left in FIG. 4B to right in FIG. 4D, the light images 321, 322, 323 and 324 of the light image module 320 having different strengths of ODEP force respectively are also moving from left to right as indicated by arrows in which deep color represents the light image having larger ODEP force, light color represents the light image having smaller ODEP force (i.e., the color depth increases as the ODEP force increases), and the color depth increases from left to right in which the color depth of the light image 321 is the greatest, the color depth of the light image 324 is the least.

As drug-susceptible microorganisms 122 moving from left in FIG. 4F to right in FIG. 4H, the light images 321, 322, 323 and 324 of the light image module 320 having different strengths of ODEP force respectively are also moving from left to right as indicated by arrows in which deep color represents the light image having larger ODEP force, light color represents the light image having smaller ODEP force (i.e., the color depth increases as the ODEP force increases), and the color depth increases from left to right in which the color depth of the light image 321 is the greatest, the color depth of the light image 324 is the least.

As shown in FIG. 4D specifically, the drug-resistant microorganisms 121 are concentrated on the right side due to its high electrical conductivity, and the images 321 and 322 having larger ODEP force than the images 323 and 324 on the left are easy to catch.

As shown in FIG. 4H specifically, the drug-susceptible microorganisms 122 are concentrated on the left side due to its low electrical conductivity, and the images 323 and 324 having smaller ODEP force than the images 321 and 322 on the right are easy to catch.

It is found that distribution and presentation of the drug-resistant microorganisms 121 (see FIG. 4D) are different from that of the drug-susceptible microorganisms 122 (see FIG. 4H).

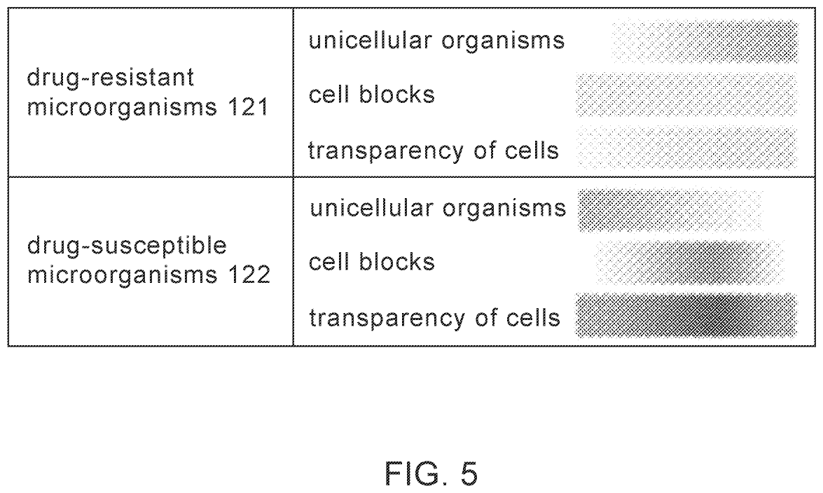

Referring to FIG. 5 in conjunction with steps S3 and S4 of FIG. 1, it is a table showing a result of analyzing both the drug-resistant microorganisms 121 in FIG. 4D and the drug-susceptible microorganisms 122 in FIG. 4H in which distributions and presentations of the microorganisms 12 are shown using color depth changes.

After the drug-resistant microorganisms 121 has been measured, regarding unicellular organisms color depth to the right is deeper than that to the left in a color bar and it means the unicellular organisms are concentrated on the right of the chip member 20; cell blocks are shown in light color in a color bar and it means almost no cell blocks; and transparency of cells is shown in light color in a color bar and it means the transparency of cells is relatively high.

After the drug-susceptible microorganisms 122 has been measured, regarding unicellular organisms color depth to the left is deeper than that to the right in a color bar and it means the unicellular organisms are concentrated on the left of the chip member 20; cell blocks are shown in deep color in a color bar and it means there are cell blocks; and transparency of cells is shown in deep color in a color bar and it means the transparency of cells is relatively low.

It is envisaged by the invention that the microorganisms 12 are classified as the drug-resistant microorganisms 121, and the drug-susceptible microorganisms 122 and the drug-resistant microorganisms 121 and the drug-susceptible microorganisms 122 are different in electrical properties After both the drug-resistant microorganisms 121 and the drug-susceptible microorganisms 122 have been analyzed by the light image module 320, they show different distributions and presentations. It is desirable to analyze the sample solution of microorganisms to be tested 14 using the light image module 320 to obtain different distributions and presentations of the microorganisms 12 which are used to understand and estimate drug resistance of the microorganisms 12. Likewise, specific microorganisms can be used to create specific modes in order to identify species, sub-species, drug resistance, toxicity, or metabolic properties of different microorganisms.

While the invention has been described in terms of preferred embodiments, those skilled in the art will recognize that the invention can be practiced with modifications within the spirit and scope of the appended claims.

What is claimed is:

1. A method of identifying properties of microorganisms using optically-induced dielectrophoresis (ODEP) force, comprising the steps of:
(a) obtaining a sample solution of microorganisms including a plurality of microorganisms having predetermined properties;
(b) pre-processing the sample solution of microorganisms to obtain a sample solution of microorganisms to be tested having predetermined electrical properties;
(c) placing the sample solution of microorganisms to be tested on a channel of a chip member;
(d) activating a light image module having combinations of different strengths of ODEP force to analyze the sample solution of microorganisms to be tested on the channel wherein the microorganisms in the sample solution of microorganisms to be tested are configured to generate a predetermined distribution and a predetermined presentation due to the predetermined electrical properties; and
(e) identifying properties of the microorganisms based on the predetermined distribution and the predetermined presentation.

2. The method of claim 1, wherein the predetermined properties of the microorganisms include species of the microorganisms, sub-species of the microorganisms, drug resistance of the microorganisms, toxicity of the microorganisms, and metabolic properties of the microorganisms.

3. The method of claim 1, wherein the light image module includes a plurality of light images, and operations of the light image module are achieved by adjusting a moving speed of the light images, a rotational speed of the light images, a spectral flux of the light images, and a shape of the light image; applying a frequency of alternating current (AC) to the chip member; applying a voltage of AC to the chip member; or any combination thereof.

4. The method of claim 1, wherein step (b) of pre-processing involves culturing microorganisms, contact force, radiation, light wave, acoustic wave, shock wave, heating, freezing, electrical wave, magnetic wave, medicine or any combination thereof.

5. The method of claim 1, wherein the chip member includes, from top to bottom, a cover, a channel layer, and a photoconductive layer; the channel layer includes the channel; and the cover includes a sample inlet aligned with a first end of the channel.

6. The method of claim 5, further comprising a projection device provided under the photoconductive layer, the projection device being configured to project light to the chip member.

7. The method of claim 1, wherein the microorganisms are bacteria, molds, rickettsia or viruses.

8. The method of claim 1, wherein the sample solution of microorganisms is obtained by culturing blood, urine, saliva, sweat, feces, pleural fluid, ascites, or cerebrospinal fluid.
